(19) United States
(12) Patent Application Publication
(10) Pub. No.: US 2023/0250458 A1
(43) Pub. Date: Aug. 10, 2023

(54) METHOD FOR PREPARING MODIFIED STARCH AS ALTERNATIVE TO HYDROXYPROPYL DISTARCH PHOSPHATE

(71) Applicant: Qilu University of Technology, Jinan (CN)

(72) Inventors: Li GUO, Jinan (CN); Bo CUI, Jinan (CN); Ruobing LI, Jinan (CN); Chao YUAN, Jinan (CN); Feixue ZOU, Jinan (CN); Wei GAO, Jinan (CN); Pengfei LIU, Jinan (CN); Die DONG, Jinan (CN); Yunlong CUI, Jinan (CN); Shuo SUN, Jinan (CN)

(21) Appl. No.: 18/302,861

(22) Filed: Apr. 19, 2023

(30) Foreign Application Priority Data
Dec. 14, 2022 (CN) 202211602372.2

(51) Int. Cl. C12P 19/04; C12P 19/14; C13K 1/06

(52) U.S. Cl. CPC C12P 19/04; C12P 19/14; C13K 1/06

(57) ABSTRACT

A method for preparing a modified starch as an alternative to hydroxypropyl distarch phosphate, including: (a) evenly mixing a cassava starch with a buffer solution to obtain a starch suspension with desired pH; (b) adding α-amylase into the starch suspension for enzymolysis of glycosidic bonds followed by deactivation to obtain a first enzymolysis product; (c) adding hexose oxidase into the first enzymolysis product for lactonization of gluco-oligosaccharides followed by deactivation to obtain a second enzymolysis product; and (d) subjecting the second enzymolysis product to centrifugation, washing and freeze drying to obtain an enzymatically-modified cassava starch.

### CROSS-REFERENCE TO RELATED APPLICATIONS

This application claims the benefit of priority from Chinese Patent Application No. 202211602372.2, filed on Dec. 14, 2022. The content of the aforementioned application, including any intervening amendments thereto, is incorporated herein by reference in its entirety.

### TECHNICAL FIELD

The present disclosure relates to starch production, in particular to a method for preparing a modified starch as an alternative to hydroxypropyl distarch phosphate.

### BACKGROUND

China is rich in starch resources. In 2020, the total starch production in China was up to 33.89 million tons. As a food additive, the starch is beneficial to the food processing, and can contribute some required properties, such as shape, taste, thickening property, gelling property, adhesion and stability, to food systems. In order to satisfy the particular processing requirements, it is often required to control and modify the inherent properties of starch. According to the structure and physicochemical properties, the starch is processed by chemical, physical or biological modification for a special application. Particularly, the chemical modification can significantly improve the physical and chemical processing properties of natural starch, and thus has been widely used in food, medicine and material fields.

Unfortunately, the chemical modification struggles with large consumption of chemical reagents, and some chemical reagents (such as propylene oxide and acetic anhydride) have high potential risk, complex storage and treatment conditions, and poor reaction stability. In view of this, the chemical modification often involves discontinuous production, serious environmental pollution, and food safety risk. Therefore, it is urgent to develop an alternative to the chemically-modified starches.

Regarding the enzymatic modification, various enzymes have been employed to process the starch to change the molecular size, structure, chain length distribution and starch paste properties, so as to form specific particle or molecular configuration. However, it has been rarely reported about the use of enzymatic modification to give a green modified starch as an alternative to the chemically-modified starch with stable properties.

Hydroxypropyl di starch phosphate has been extensively used as a thickening agent, suspending agent or coating in food and chemical industry. The hydroxypropyl distarch phosphate is stable in paste viscosity, and thus is suitable for the processing and production of the frozen foods and convenience foods. The hydroxypropyl distarch phosphate can contribute good water retention to food during the low-temperature storage and transportation, and can lead to enhanced heat resistance, acid resistance and shear resistance. Moreover, as the thickening agent, the hydroxypropyl distarch phosphate can make the gravies, sauces, juices, and puddings smooth, thick, transparent, clear, and free of granular structures, and have good freeze-thaw stability, boiling resistance and good taste. However, the hydroxypropyl di starch phosphate has complex preparation and poor reaction stability, and the raw material propylene oxide is explosive and harmful, and thus requires strict storage conditions. Moreover, the propylene oxide will result in complex sewage treatment and serious environmental pollution. Accordingly, an enzymatically-modified starch having high additional value, excellent properties, green preparation and good safety is provided to replace the hydroxypropyl distarch phosphate, which is prepared by modifying cassava starch under the catalysis of α-amylase and hexose oxidase, promoting the sustainable development of modified starches.

### SUMMARY

In order to overcome the problems in the prior art, this application provides a simple, safe, green and economic method for preparing a modified starch as an alternative to hydroxypropyl distarch phosphate.

Technical solutions of this application are described as follows.

This application provides a method for preparing a modified starch as an alternative to hydroxypropyl distarch phosphate, comprising:

(a) evenly mixing a cassava starch with a buffer solution followed by pH adjustment to obtain a starch suspension;

(b) adding α-amylase into the starch suspension for enzymatic hydrolysis of glycosidic bonds followed by deactivation to obtain a first enzymolysis product;

(c) adding hexose oxidase into the first enzymolysis product to catalyze lactonization of gluco-oligosaccharides in the first enzymolysis product followed by deactivation to obtain a second enzymolysis product; and

(d) subjecting the second enzymolysis product to centrifugation, washing and freeze drying to obtain the modified starch.

Under the catalysis of the α-amylase, α-1, 4-glycosidic bonds of the starch are partially hydrolyzed to generate products including dextrin, glucan oligosaccharides and hexose. Then the glucan oligosaccharides varying in molecular weight are oxidized in the presence of the hexose oxidase under an aerobic environment to generate corresponding lactone substances. The chemical properties of the lactone substances, such as paste viscosity, freeze-thaw stability, shear resistance and ageing resistance, are similar to or better than those of hydroxypropyl distarch phosphate.

In some embodiments, step (a) is performed through steps of:

mixing 10-30 g of the cassava starch with 200-400 mL of a 0.01M disodium hydrogen phosphate-sodium dihydrogen phosphate buffer solution followed by adjustment to pH 4.0-6.0 and stirring in a water bath at 40-60° C. to obtain the starch suspension.

In some embodiments, step (a) is performed through steps of: mixing 20 g of the cassava starch with 200 mL of the 0.01 M disodium hydrogen phosphate-sodium dihydrogen phosphate buffer solution followed by adjustment to pH 6.0 and stirring in a water bath at 45° C. to obtain the starch suspension.

In some embodiments, in step (b), the α-amylase has an activity of 20-40 U/g; the α-amylase is 2.5-5.5% by weight of the cassava starch on dry basis; the enzymatic hydrolysis is performed for 30-120 min; and the deactivation is performed by adjusting pH to 9-10 with a 1 mol/L sodium hydroxide solution.

In some embodiments, in step (b), the α-amylase has an activity of 20 U/g; the α-amylase is 5% by weight of the cassava starch on dry basis; the enzymatic hydrolysis is performed for 30 min; and the deactivation is performed by adjusting pH to 9.5 with the 1 mol/L sodium hydroxide solution.

In some embodiments, step (c) further comprises:

adjusting the first enzymolysis product to pH 5.0-7.0 with 0.01 M disodium hydrogen phosphate-sodium dihydrogen phosphate buffer solution;

wherein the lactonization is performed under stirring at 20-40° C. in a water bath for 1-3 h; the hexose oxidase is 1.5-5% by weight of the cassava starch on dry basis; the hexose oxidase has an activity of 20-60 U/g; and the deactivation is performed by adjusting pH to 9-10 with a 1 mol/L sodium hydroxide solution.

In some embodiments, in step (c), the first enzymolysis product is adjusted to pH 6.0; the lactonization is performed under stirring at 25° C. in a water bath for 1 h; the hexose oxidase is 5% by weight of the cassava starch on dry basis; the hexose oxidase has an activity of 20 U/g; and the deactivation is performed by adjusting pH to 10 with the 1 mol/L sodium hydroxide solution.

In some embodiments, step (d) is performed through steps of:

centrifuging the second enzymolysis product at 5,000-8,000 rpm for 10-20 min to collect a first precipitate;

washing the first precipitate three times with water followed by centrifugation at 5,000-8,000 rpm for 10-20 min to collect a second precipitate, wherein a volume ratio of water for each washing to the second enzymolysis product is 3:1; and

subjecting the second precipitate to freeze drying to obtain the modified starch.

In some embodiments, in step (d), the second enzymolysis product is centrifuged at 8,000 rpm for 10 min to collect the first precipitate; and the first precipitate is washed three times with water, and centrifuged at 6,000 rpm for 10 min to collect the second precipitate.

Compared to the prior art, this application has the following beneficial effects.

This application provides an alternative to hydroxypropyl distarch phosphate, which is prepared by modifying the cassava starch by means of the α-amylase and the hexose oxidase, overcoming the defects of high safety risk and serious environmental pollution in the preparation of chemically-modified starches. The green and safe enzymatically-modified starch developed herein can be an ideal alternative to the hydroxypropyl distarch phosphate, thereby facilitating promoting the sustainable development of modified starch industry.

The chemical properties of the enzymatically-modified starch, such as paste viscosity, freeze-thaw stability, shear resistance and aging resistance, are similar to or better than those of hydroxypropyl distarch phosphate. Furthermore, the method provided herein is simple, eco-friendly, safe, low-cost, and pollution-free, and thus suitable for industrial production.

### BRIEF DESCRIPTION OF THE DRAWINGS

The embodiments of this application will be described in detail below with reference to the accompanying drawings.

FIG. 1 shows transparency and viscosity of a 5 wt. % paste of enzymatically-modified cassava starch, a 5 wt. % paste of an enzymatically-modified pea starch, a 5 wt. % paste of enzymatically-modified rice starch and a 5 wt. % paste of hydroxypropyl distarch phosphate; and

FIG. 2 shows freeze-thaw stability of the 5 wt. % paste of enzymatically-modified cassava starch, the 5 wt. % paste of an enzymatically-modified pea starch, the 5 wt. % paste of enzymatically-modified rice starch and the 5 wt. % paste of hydroxypropyl distarch phosphate.

### DETAILED DESCRIPTION OF EMBODIMENTS

In order to clearly illustrate the objects and technical solutions of the present invention, the present disclosure will be described below in detail with reference to the embodiments and drawings. It should be noted that described below are merely illustrative of the present disclosure, and not intended to limit the present disclosure.

### Example 1 Preparation of Modified Cassava Starch

(S1) 20 g of cassava starch was mixed with 200 mL of 0.01M disodium hydrogen phosphate-sodium dihydrogen phosphate buffer solution. The mixture was adjusted to pH 6.0, and stirred in a water bath at 45° C. to obtain a starch suspension.

(S2) 1 g of α-amylase (20 U/g) was added to the starch suspension for enzymatic hydrolysis for 30 min. The enzymolysis system was adjusted to pH 9.5 with 1 mol/L sodium hydroxide solution to terminate the enzymatic reaction, so as to obtain a first enzymolysis solution.

(S3) The first enzymolysis solution was adjusted to pH 6.0 with 0.01M disodium hydrogen phosphate-sodium dihydrogen phosphate buffer solution, added with 1 g of hexose oxidase (20 U/g) and reacted under stirring in a water bath at 25° C. for 1 h. The enzymolysis mixture was then adjusted to pH 10 with 1 mol/L sodium hydroxide solution to terminate the enzymatic reaction, so as to obtain a second enzymolysis solution.

(S4) The second enzymolysis solution was centrifuged at 8,000 rpm for 10 min, washed three times with water and centrifuged at 6,000 rpm for 10 min to collect a precipitate, where a volume ratio of water for each washing to the second enzymolysis solution was 3:1. The precipitate was freeze-dried to obtain an enzymatically-modified cassava starch (89.24% yield).

A 5 wt. % paste of the enzymatically-modified cassava starch prepared herein had a peak viscosity of 552 cP, and a freeze-thaw stability (measured as syneresis rate) of 43.981%. Comparatively, a 5 wt. % paste of hydroxypropyl distarch phosphate had a peak viscosity of 515 cP, and a freeze-thaw stability of 42.361%. In addition, a breakdown viscosity (characterizing the shear resistance) (152 cP) of the 5 wt. % paste of the enzymatically-modified cassava starch was superior to that of the 5 wt. % paste of hydroxypropyl distarch phosphate (42 cP), indicating that the shear resistance of the 5 wt. % paste of the enzymatically-modified cassava starch was better than that of the 5 wt. % paste of hydroxypropyl distarch phosphate. A setback viscosity (characterizing the aging degree) (167 cP) of the 5 wt. % paste of the enzymatically-modified cassava starch was less than that of the 5 wt. % paste of hydroxypropyl distarch phosphate (302 cP), indicating that the ageing resistance of the 5 wt. % paste of the enzymatically-modified cassava starch was better than that of the 5 wt. % paste of hydroxypropyl distarch phosphate.

### Example 2 Preparation of Modified Cassava Starch

(S1) 10 g of cassava starch was mixed with 200 mL of 0.01M disodium hydrogen phosphate-sodium dihydrogen phosphate buffer solution. The mixture was adjusted to pH 5.0, and stirred in a water bath at 50° C. to obtain a starch suspension.

(S2) 0.3 g of α-amylase (30 U/g) was added to the starch suspension for enzymatic hydrolysis for 60 min. The enzymolysis system was adjusted to pH 10 with 1 mol/L sodium hydroxide solution to terminate the enzymatic reaction, so as to obtain a first enzymolysis solution.

(S3) The first enzymolysis solution was adjusted to pH 5.8 with 0.01 M disodium hydrogen phosphate-sodium dihydrogen phosphate buffer solution, added with 0.3 g of hexose oxidase (30 U/g) and reacted under stirring in a water bath at 35° C. for 2 h. The enzymolysis mixture was then adjusted to pH 9 with 1 mol/L sodium hydroxide solution to terminate the enzymatic reaction, so as to obtain a second enzymolysis solution.

(S4) The second enzymolysis solution was centrifuged at 8,000 rpm for 10 min, washed three times with water and centrifuged at 8,000 rpm for 10 min to collect a precipitate, where a volume ratio of water for each washing to the second enzymolysis solution was 3:1. The precipitate was freeze-dried to obtain an enzymatically-modified cassava starch (85.18% yield).

A 5 wt. % paste of the enzymatically-modified cassava starch prepared herein had a peak viscosity of 682 cP, a freeze-thaw stability of 45.112%, a breakdown viscosity of 176 cP, and a setback viscosity of 207 cP.

### Example 3 Preparation of Modified Cassava Starch

(S1) 30 g of cassava starch was mixed with 400 mL of 0.01 M disodium hydrogen phosphate-sodium dihydrogen phosphate buffer solution. The mixture was adjusted to pH 4.0, and stirred in a water bath at 60° C. to obtain a starch suspension.

(S2) 102 g of α-amylase (40 U/g) was added to the starch suspension for enzymatic hydrolysis for 120 min. The enzymolysis system was adjusted to pH 10 with 1 mol/L sodium hydroxide solution to terminate the enzymatic reaction, so as to obtain a first enzymolysis solution.

(S3) The first enzymolysis solution was adjusted to pH 7.0 with 0.01M disodium hydrogen phosphate-sodium dihydrogen phosphate buffer solution, added with 1.2 g of hexose oxidase (50 U/g) and reacted under stirring in a water bath at 40° C. for 2 h. The enzymolysis mixture was then adjusted to pH 9 with 1 mol/L sodium hydroxide solution to terminate the enzymatic reaction, so as to obtain a second enzymolysis solution.

(S4) The second enzymolysis solution was centrifuged at 8,000 rpm for 20 min, washed three times with water and centrifuged at 6,000 rpm for 10 min to collect a precipitate, where a volume ratio of water for each washing to the second enzymolysis solution was 3:1. The precipitate was freeze-dried to obtain an enzymatically-modified cassava starch (80.65% yield).

A 5 wt. % paste of the enzymatically-modified cassava starch prepared herein had a peak viscosity of 490 cP, a freeze-thaw stability of 44.102%, a breakdown viscosity of 110 cP, and a setback viscosity of 174 cP.

### Comparative Example 1 Preparation of Modified Rice Starch

(S1) 20 g of rice starch was mixed with 200 mL of 0.01M disodium hydrogen phosphate-sodium dihydrogen phosphate buffer solution. The mixture was adjusted to pH 6.0, and stirred in a water bath at 45° C. to obtain a starch suspension.

(S2) α-amylase (20 U/g) was added to the starch suspension for enzymatic hydrolysis for 30 min. The enzymolysis system was adjusted to pH 9.5 with 1 mol/L sodium hydroxide solution to terminate the enzymatic reaction, so as to obtain a first enzymolysis solution.

(S3) The first enzymolysis solution was adjusted to pH 6.0 with 0.01M disodium hydrogen phosphate-sodium dihydrogen phosphate buffer solution, added with hexose oxidase (20 U/g) and reacted under stirring in a water bath at 25° C. for 1 h. The enzymolysis mixture was then adjusted to pH 9 with 1 mol/L sodium hydroxide solution to terminate the enzymatic reaction, so as to obtain a second enzymolysis solution.

(S4) The second enzymolysis solution was centrifuged at 8,000 rpm for 10 min, washed three times with water and centrifuged at 6,000 rpm for 10 min to collect a precipitate, where a volume ratio of water for each washing to the second enzymolysis solution was 3:1. The precipitate was freeze-dried to obtain an enzymatically-modified rice starch (82.98% yield).

A 5 wt. % paste of the enzymatically-modified rice starch prepared herein had a peak viscosity of 205 cP, a freeze-thaw stability of 69.332%, a breakdown viscosity of 123 cP, and a setback viscosity of 84.00 cP.

### Comparative Example 2 Preparation of Modified Pea Starch

(S1) 20 g of pea starch was mixed with 200 mL of 0.01M disodium hydrogen phosphate-sodium dihydrogen phosphate buffer solution. The mixture was adjusted to pH 6.0, and stirred in a water bath at 45° C. to obtain a starch suspension.

(S2) α-amylase (20 U/g) was added to the starch suspension for enzymatic hydrolysis for 30 min. The enzymolysis system was adjusted to pH 9.5 with 1 mol/L sodium hydroxide solution to terminate the enzymatic reaction, so as to obtain a first enzymolysis solution.

(S3) The first enzymolysis solution was adjusted to pH 6.0 with 0.01M disodium hydrogen phosphate-sodium dihydrogen phosphate buffer solution, added with hexose oxidase (20 U/g) and reacted under stirring in a water bath at 25° C. for 1 h. The enzymolysis mixture was then adjusted to pH 10 with 1 mol/L sodium hydroxide solution to terminate the enzymatic reaction, so as to obtain a second enzymolysis solution.

(S4) The second enzymolysis solution was centrifuged at 8,000 rpm for 10 min, washed three times with water and centrifuged at 6,000 rpm for 10 min to collect a precipitate, where a volume ratio of water for each washing to the second enzymolysis solution was 3:1. The precipitate was freeze-dried to obtain an enzymatically-modified cassava starch (86.11% yield).

A 5 wt. % paste of the enzymatically-modified pea starch prepared herein had a peak viscosity of 126 cP, a freeze-thaw stability of 72.636%, a breakdown viscosity of 7 cP, and a setback viscosity of 56 cP.

Table 1 showed properties of several enzymatically-modified starch pastes and hydroxypropyl distarch phosphate paste.

TABLE 1
Properties and freeze-thaw stability of enzymatically-modified starch pastes
and hydroxypropyl distarch phosphate paste

| | | | | | | | Freeze- |
|---|---|---|---|---|---|---|---|
| | Peak | Trough | Breakdown | Final | Setback | Pasting | thaw |
| | viscosity | viscosity | viscosity | viscosity | viscosity | temperature | stability |
| Sample | (cP) | (cP) | (cP) | (cP) | (cP) | (° C.) | (%) |
| Commercially- | 515 | 468 | 47 | 770 | 302 | 66.1 | 42.361 |
| available | | | | | | | |
| Hydroxypropyl | | | | | | | |
| distarch phosphate | | | | | | | |
| paste | | | | | | | |
| Enzymatically- | 552 | 400 | 152 | 567 | 167 | 74.3 | 43.981 |
| modified cassava | | | | | | | |
| starch paste | | | | | | | |
| (Example 1) | | | | | | | |
| Enzymatically- | 205 | 123 | 82 | 399 | 216 | 84.0 | 69.332 |
| modified rice | | | | | | | |
| starch paste | | | | | | | |
| (Comparative | | | | | | | |
| Example 1) | | | | | | | |
| Enzymatically- | 126 | 119 | 7 | 175 | 56 | 76.7 | 72.636 |
| modified pea | | | | | | | |
| starch paste | | | | | | | |
| (Comparative | | | | | | | |
| Example 2) | | | | | | | |

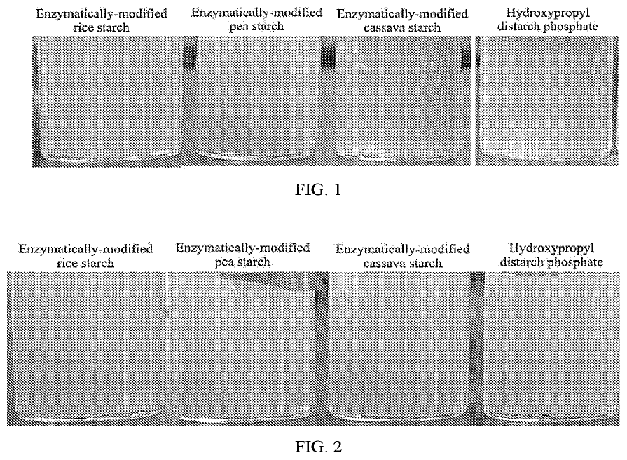

FIG. 1 illustrated comparison of the 5 wt. % paste of the enzymatically-modified cassava starch prepared in Example 1, the 5 wt. % paste of the enzymatically-modified pea starch, the 5 wt. % paste of the enzymatically-modified rice starch and the 5 wt. % paste of the hydroxypropyl distarch phosphate in transparency and viscosity.

FIG. 2 illustrated comparison of the 5 wt. % paste of the enzymatically-modified cassava starch prepared in Example 1, the 5 wt. % paste of the enzymatically-modified pea starch, the 5 wt. % paste of the enzymatically-modified rice starch and the 5 wt. % paste of the hydroxypropyl distarch phosphate in freeze-thaw stability.

A method for preparing a modified starch by using the α-amylase and the hexose oxidase was provided herein. The chemical properties of enzymatically-modified cassava starch paste, such as paste viscosity, freeze-thaw stability, shear resistance and ageing resistance, are close to or better than those of hydroxypropyl distarch phosphate. Regarding the preparation of the hydroxypropyl distarch phosphate, costs for the reagents including propylene oxide, sodium trimetaphosphate, acid liquor and alkali liquor are about 3000 RMB per ton, furthermore, these reagents led to by-products such as salt compounds. Regarding the method provided herein, the costs for the α-amylase and the hexose oxidase are about 3000 RMB per ton, and merely a small number of by-products including glucose, maltose, maltobiose and maltotriose would be generated. The method provided herein is simple, eco-friendly and safe, and a product has stable property.

Described above are merely preferred embodiments of the disclosure, which are not intended to limit the disclosure. It should be understood that variations made by those skilled in the art without departing from the spirit and scope of the disclosure shall fall within the scope of the disclosure defined by the appended claims.

What is claimed is:

1. A method for preparing a modified starch, comprising:
(a) evenly mixing a cassava starch with a buffer solution followed by pH adjustment to obtain a starch suspension;
(b) adding α-amylase into the starch suspension for enzymatic hydrolysis of glycosidic bonds followed by deactivation to obtain a first enzymolysis product;
(c) adding hexose oxidase into the first enzymolysis product to catalyze lactonization of gluco-oligosaccharides in the first enzymolysis product followed by deactivation to obtain a second enzymolysis product; and
(d) subjecting the second enzymolysis product to centrifugation, washing and freeze drying to obtain the modified starch.

2. The method of claim 1, wherein step (a) is performed through steps of:
mixing 10-30 g of the cassava starch with 200-400 mL of a 0.01M disodium hydrogen phosphate-sodium dihydrogen phosphate buffer solution followed by adjustment to pH 4.0-6.0 and stirring in a water bath at 40-60° C. to obtain the starch suspension.

3. The method of claim 1, wherein in step (b), the α-amylase has an activity of 20-40 U/g; the α-amylase is 2.5-5.5% by weight of the cassava starch on dry basis; the enzymatic hydrolysis is performed for 30-120 min; and the deactivation is performed by adjusting pH to 9-10 with a 1 mol/L sodium hydroxide solution.

4. The method of claim 1, wherein step (c) further comprises:
adjusting the first enzymolysis product to pH 5.0-7.0 with 0.01M disodium hydrogen phosphate-sodium dihydrogen phosphate buffer solution;
wherein the lactonization is performed under stirring at 20-40° C. in a water bath for 1-3 h; the hexose oxidase is 1.5-5% by weight of the cassava starch on dry basis; the hexose oxidase has an activity of 20-60 U/g; and the deactivation is performed by adjusting pH to 9-10 with a 1 mol/L sodium hydroxide solution.

5. The method of claim 1, wherein step (d) is performed through steps of:
centrifuging the second enzymolysis product at 5,000-8,000 rpm for 10-20 min to collect a first precipitate;
washing the first precipitate three times with water followed by centrifugation at 5,000-8,000 rpm for 10-20 min to collect a second precipitate, wherein a volume ratio of water for each washing to the second enzymolysis product is 3:1; and
subjecting the second precipitate to freeze drying to obtain the modified starch.

6. The method of claim 2, wherein step (a) is performed through steps of:
mixing 20 g of the cassava starch with 200 mL of the 0.01M disodium hydrogen phosphate-sodium dihydrogen phosphate buffer solution followed by adjustment to pH 6.0 and stirring in a water bath at 45° C. to obtain the starch suspension.

7. The method of claim 3, wherein in step (b), the α-amylase has an activity of 20 U/g; the α-amylase is 5% by weight of the cassava starch on dry basis; the enzymatic hydrolysis is performed for 30 min; and the deactivation is performed by adjusting pH to 9.5 with the 1 mol/L sodium hydroxide solution.

8. The method of claim 4, wherein in step (c), the first enzymolysis product is adjusted to pH 6.0; the lactonization is performed under stirring at 25° C. in a water bath for 1 h; the hexose oxidase is 5% by weight of the cassava starch on dry basis; the hexose oxidase has an activity of 20 U/g; and the deactivation is performed by adjusting pH to 10 with the 1 mol/L sodium hydroxide solution.

9. The method of claim 5, wherein in step (d), the second enzymolysis product is centrifuged at 8,000 rpm for 10 min to collect the first precipitate; and the first precipitate is washed three times with water, and centrifuged at 6,000 rpm for 10 min to collect the second precipitate.
